(19) United States
(12) Patent Application Publication
(10) Pub. No.: US 2002/0142945 A1
(43) Pub. Date: Oct. 3, 2002

(54) Immunomodulatory methods using carbohydrate antigens

(75) Inventors: Donald A. Harn, Pembroke, MA (US); Mitsuhiro Okano, Okayama-city (JP)

Correspondence Address: LAHIVE & COCKFIELD, 28 STATE STREET, BOSTON, MA, 02109, US

(73) Assignee: President and Fellows of Harvard College

(21) Appl. No.: 10/024,730

(22) Filed: Dec. 19, 2001

Related U.S. Application Data
(63) Continuation of application No. 09/451,745, filed on Nov. 30, 1999, now abandoned.
(60) Provisional application No. 60/110,496, filed on Dec. 1, 1998.

(51) Int. Cl. A61K 38/38; A61K 31/721; A61K 31/715

(52) U.S. Cl. 514/8; 514/61; 514/59

(57) ABSTRACT

Methods for modulating immune responses, in particular IgE responses, are provided. The methods involve contacting an cell with an agent comprising multivalent lacto-N-neotetraose (LNnT), which modulates an immune response. The methods are useful for enhancing production of non-specific polyclonal IgE, inhibiting production of antigen-specific IgE responses, inducing cytokine production, and stimulating proliferation of splenocytes. In a preferred embodiment, the invention provides methods for modulating an immune response to an antigen (e.g., an allergan) in vivo. Pharmaceutical compositions for modulating immune responses comprising the agents of the invention are also provided.

### BACKGROUND OF THE INVENTION

Helminth infection is strongly associated with the production of large amounts of serum IgE in humans and experimental animals (King, C L. et al., (1993), J. Immunol. vol. 150 pp. 1873-1880; Ogilvie B M, Nature 1964 204. 91-92; Sadun, E H. et al. (1970) Exp. Parasitol. vol. 28 pp. 435-449). The majority of serum IgE in infected host is nonspecific and polyclonal (Dessaint, J P. et al. (1975) Clin. Exp. Immunol. vol. 20 pp. 427-436; Jarret, et al. (1976) Clin. Exp. Immunol. vol. 24 pp. 346-351). Such IgE has several roles in immunity against helminth parasites. Ag-specific IgE, especially anti-adult worm Ag, is involved with the resistance to reinfection (Hagan, P. et al. (1991) Nature vol. 349 pp. 243-245; Viana, I R C. et al. (1995) Parasite Immunol. vol. 17 pp. 297-304), and cell-mediated cytotoxicity against parasites (Capron, A. et al. (1980) Am. J. Trop. Med. Hyg. vol. 29 pp. 849-857; Gounni, A S. et al. (1994) Nature vol. 367 pp. 183-186; Cutts, L. et al. (1997) Parasite Immunol. vol. 19 pp. 91-102). On the other hand, non-specific, polyclonal IgE seems to be beneficial for both host and parasites to reduce the risk of the potentially lethal anaphylaxic reaction to parasitic antigens by saturating the available Fc_{ε}Rs on effector cells, although IgE plays a detrimental role for the host in primary infection of schistosomiasis (Hagan, P. (1993) Parasite Immunol. vol. 15 p. 14; Pritchard, D I. (1993) Parasite Immunol. vol. 15 pp. 5-9; Amiri, P. et al. (1993) J. Exp. Med. vol. 180 pp. 43-51).

Several allergic components in schistosomal Ag have been reported to react with IgE from humans and rodents at various stages of infections. (Damonneville, M. et al. (1984) Int. Arch. Allergy appl. Immunol. vol. 73 pp. 248-255; Owhashi, M. et al. (1986) Int. Arch. Allergy appl. Immunol. vol. 81 pp. 129-135). However little is known about the molecules that induce polyclonal IgE production from host. Toxocara canis adult worm antigen has B cell mitogenic activity to induce proliferation, IgG and IgE production although the allergenic molecule remains undefined (Wang, M Q. et al. (1995) Parasite Immunol. vol. 17 pp. 609-615.Yamashita, U. et al. (1993) Jap. J. Parasitol. vol. 42 pp. 211-219). Ascaris body fluid also contains a B cell mitogen, however, this mitogen needs the help of IL4 delivered by the other factors to induce polyclonal IgE production (McGibbon, A M. et al. (1990) Mol. Biochem. Parasitol. vol. 39 pp. 163-172; Lee, T D G. et al. (1995) J. Allergy Clin. Immunol. vol. 95 pp. 124-1254.Lee, T D G. et al. (1993) Int. Arch. Allergy Immunol. vol. 102 pp. 185-190). Two recombinant filarial proteins are capable of inducing polyclonal IgE production in vitro, however, they also induce Ag-specific IgE (Garrud, O. et al. (1995) J. Immunol. vol. 155 pp. 131-1325).

Schistosoma mansoni synthesize glycoproteins containing polylactosamine sugars (Srivastan, J. et al. (1992) J. Biol. Chem. vol. 267 pp. 14730-14737; Van Dam, G J. et al. (1994) Eur. J. Biochem. vol. 225 pp. 467-482). Lacto-N-fucopentaose III (LNFIII) found on adult worm and egg of S. mansoni has been found to be an antigenic determinant (KO, A I. et al. (1990) Proc. Natl. Acad. Sci. USA vol. 87 pp. 4159-4163). LNFIII stimulates splenic B cells from parasite-infected mice to proliferate and produce IL-10, a cytokine that downregulates Th1 immune responses (W Velupillai, P. et al. Proc. Natl. Acad. Sci. USA vol. 91 pp. 18-22; Palanivel, V. et al. (1996) Exp. Parasitol. vol. 84 pp. 168-177; Velupillai, P. et al. (1997) J. Immunol. vol. 158 pp. 338-344). In addition, this sugar has been found to have an adjuvant effect in inducing Th2 immune reaction in both Ab and cytokine production against protein with which the sugar is physically conjugated.

Additional insight into molecules which bias the immune response towards specific cytokine profiles will be important in developing methods of regulating immune responses. In particular, identification of molecules which induce polyclonal IgE synthesis will be of tremendous benefit in treating allergy.

### SUMMARY OF THE INVENTION

The instant invention provides novel methods of regulating the immune response. The invention is based, at least in part, on the discovery of the functional characteristics of LNnT, a nonfucosylated homologue of LNFIII, that is converted to LNFIII by α1-3 fucosyltransferase in adult worms. Multivalent LNnT induces polyclonal IgE production and cytokine production from mice by IP inoculation. This molecule can be used to modulate the IgE response, not only to parasite antigens, but to environmental allergens by saturating Fcε Rs on the effector cells.

### BRIEF DESCRIPTION OF THE DRAWINGS

FIGS. 1a-1c: Serum Ig production in BALB/C mice following IP immunization.

FIG. 1(a): Female BALB/C mice were IP immunized with saline, HSA, Le^{y}-HSA, LNnT-HSA or HSA-Alum. All the antigens were inoculated at the dose of 10 μg protein weight of HSA. 6 and 5 days following first (dotted bar) and second (closed bar) boosting immunization, respectively, blood samples were taken and serum total IgE was measured. Results shown are the mean±1 SE of four individual serum.

FIG. 1(b): Course of serum total IgE following IP immunization. BALB/C mice were IP immunized with saline (open square), HSA (10 μg; open triangle) or LNnT-HSA (10 μg of HSA; closed circle) at day 0. Two weeks later, the boosting immunization was followed in a same fashion. Blood was taken at day 10, 20, 27, 41, and 69, and serum total IgE was determined. Results shown are the mean±1 SE of four individual serum.

FIG. 1(c): Serum IgG isotypes 5 days following third IP immunization with saline (dotted bar), HSA (open bar), or LNnT-HSA (closed bar). Results shown are the mean±1 SE of four individual serum. All the results are representative of three experiments.

FIGS. 2a-2d Antigen-specific Ab production following IP immunization with multivalent sugars. Female BALB/C mice were immunized and bled as described in FIG. 1. Following first (dotted bar) and second (closed bar) immunization, HSA-specific IgE (FIG. 2a), HSA-specific IgG (FIG. 2b), LNnT-HSA-specific IgE (FIG. 2c), and LNnT-HSA-specific IgG (FIG. 2d) were measured by ELISA as described in materials and methods. Specific IgE were determined as the absorbance at 450 nm of sera diluted 4 times. Specific IgG were determined as the endpoint titer. Results shown are the mean±1 SE of four individual serum. Results are representative of three experiments.

FIGS. 3a-3d: Serum total IgE in CBA/J (FIG. 3a) and C57BL/6 (FIG. 3b) mice following IP immunization with saline, HSA or LNnT-HSA. 6 and 5 days following first (dotted bar) and second (closed bar) boosting immunization, respectively, blood samples were taken and serum total IgE were determined. Results shown are the mean±1 SE of four individual serum.

FIG. 3c: Course of serum total IgE following SC immunization. BALB/C mice were SC immunized with saline (open square), HSA (10 μg; open triangle) or LNnT-HSA (10 μg of HSA; closed circle) at day 0. Two weeks later, the boosting immunization was followed in a same fashion. Blood was taken at day 10, 20, 27, 41, and 69, and serum total IgE was determined. Results shown are the mean±1 SE of four individual serum.

FIG. 3D: Serum total IgE in CBA/CaJ (open bar) and CBA/CaJ xid (hatched bar) following second boosting IP immunization with saline, HSA or LNnT-HSA. Results shown are the mean±1 SE of four individual serum. Results are representative of two experiments.

FIG. 4 Proliferative responses of splenocytes. BALB/C mice were IP immunized with saline, HSA (10 μg) or LNnT-HSA (0 μg weight of HSA). 2 and 3 weeks later, boosting immunization was done in the same fashion. 5 days following the final immunization, spleens were removed. 2.5×10⁶ splenocytes were stimulated with ConA (2 μg/ml; dotted bar), HSA (10 μg/ml; hatched bar), LNnT-HSA (10 μg/ml of HSA; closed bar) or no restimulation (open bar). Results shown are the mean cpm (experimental-medium control)±1 SE of four individual mice per group. Results are representative of three experiments.

FIGS. 5a-5h: B7-1 and B7-2 expression on B220+ cells. Splenocytes from mice IP immunized with saline (FIGS. 5a, 5d), HSA (FIGS. 5b, 5e), or LNnT-HSA (FIGS. 5c, 5f) were incubated for 24 hrs without additional stimulants. Cell pellets were stained with PE-conjugated mAb against either B7-l (FIGS. 5a, 5b, 5c) or B7-2 (FIGS. 5d, 5e, 5f). Numbers expressed in upper right quadrant show the mean±1 SE of percentage of cells expressing both B220 and B7 molecules from six individual sample. FIGS. 5g, 5h: Effect of the duration of culture incubation on B7 expression in mice IP immunized with saline (open square), HSA (open triangle) or LNnT-HSA (closed circle). Percentage of B220+ cells expressing B7-1 (FIG. 5g) or B7-2 (FIG. 5h) were monitored at preincubation, 24, 72, and 120 hrs postincubation without restimulation. Results were representative for two experiments.

FIG. 6: Levels of total IgE (ng/ml) in mice treated two (open bars) or three (closed bars) times intraperitoneally with the LNnT-dextran conjugate LNnT35.

FIG. 7: Levels of total IgE (ng/ml) in mice treated first with RMPI (saline), ovalbumin, the LNnT-dextran conjugate LNnT45, or dextran followed by booster treatment with ovalbumin.

FIG. 8: Levels of ovalbumin-specific IgE in mice treated first with RMPI (saline), ovalbumin, the LNnT-dextran conjugate LNnT45, or dextran followed by booster treatment with ovalbumin.

FIG. 9: Levels of total IgE (ng/ml) over time (up to 70 days post-immunization) in mice treated with either saline, HSA or LNnT-HSA.

FIGS. 10a-10c: Levels of Th2-type cytokine production by total spleen cells of mice immunized with vehicle (dextran) o r LNnT-dextran conjugate. FIG. 10a shows IL-13 levels (pg/ml). FIG. 10b shows IL-4 levels (pg/ml). FIG. 10c shows IL-10 levels (pg/ml).

FIG. 11: Level of production of the Th1-type cytokine interferon-gamma (pg/ml) by total spleen cells of mice immunized with vehicle (dextran) or LNnT-dextran conjugate.

### DETAILED DESCRIPTION OF THE INVENTION

This invention provides immunomodulatory methods in which a cell (e.g., a human immune cell) is contacted with an agent which modulates an immune response (e.g., non-specific polyclonal IgE production, immune cell mitogenesis, or production by the cell of one or more cytokines). The invention is based, at least in part, on the discovery that when animals are immunized with multivalent lacto-N-neotetraose (LNnT), a carbohydrate that is putatively expressed on helminth parasite Schistosoma mansosi, both BALB/C and CBA/J mice produced significantly higher amounts of total serum IgE following two intraperitoneal (IP) immunizations with multivalent LNnT conjugated to human serum albumin (LNnT-HSA) compared to groups immunized with saline or HSA alone. Interestingly, no specific IgE against the carbohydrate or the carrier protein was detected in ELISA, suggesting an induction of polyclonal nonspecific IgE production in vivo by multivalent LNnT. Moreover, this neo-glycoprotein did not promote significant production of IgG against either the carbohydrate or the carrier protein. C57BL/6 mice only showed the elevation of serum total IgE after three times immunization with LNnT-HSA, reflecting strain-dependent reactions against LNnT-HSA. Spleen cells from mice IP immunized with LNnT-HSA produced in vitro significant amount of IL-4, IL-5, and IL-10 as well as IL-2 and IFN-γ compared to controls. In addition, cultured B220+ cells had increased expression of B7-2 (CD86) but not B7-1 (CD80), suggesting that B7-2 expression is strongly associated with polyclonal production of IgE. Further, IL-4 gene deficient BALB/C mice did not produce polyclonal IgE following IP immunization with LNnT-HSA. Spleen cells from these mice produced lower but significant amounts of IL-5 and IL-10, and same amounts of IFN-γ compared to the wild type, demonstrating that IL-4 is critical for promoting polyclonal IgE production induced by the multivalent carbohydrate naturally expressed on helminth parasite.

Additional experiments demonstrated that LNnT conjugated to dextran also stimulates polyclonal IgE responses and that pretreatment with multivalent LNnT (e.g., LNnT conjugated to dextran), prior to immunization with an antigen, inhibits the production of antigen-specific IgE responses. The enhanced levels of polyclonal IgE stimulated by multivalent LNnT are persistent (e.g., sustained for at least 70 days). In addition to IL-4, the production of Th2-type cytokines IL-10 and IL-13 is stimulated by treatment with LNnT conjugated to dextran, whereas levels of the Th1-type cytokine interferon gamma are inhibited by treatment with LNnT conjugated to dextran.

Thus, the methods of the invention allow for IgE production to be modulated (e.g., stimulation of non-specific IgE and/or inhibition of antigen-specific IgE), as well as allowing for modulation of cytokine production. Accordingly, the immunomodulatory methods of the invention allow for an immune response to be biased towards a specific cytokine secretion profile, for example, a Th2 response. The ability to influence the production of non-specific, polyclonal IgE using the immunomodulatory methods of the invention can be used in the prevention of detrimental host reaction against parasite infection and is further applicable to the protection against environmental allergens by saturating FcεRs on effector cells. Moreover, the ability to influence the development of, for example, a Th2 response using the immunomodulatory methods of the invention is applicable to the treatment of a wide variety of disorders, including cancer, infectious diseases (e.g., HIV and tuberculosis), allergies and autoimmune diseases.

In order that the present invention may be more readily understood, certain terms are first defined. Standard abbreviations for sugars are used herein.

As used herein, the term “lacto-N-neotetraose” (“LNn”) is intended to refer to a polylactosamine sugar which is a non-fucosylated homologue of lacto-N-fucopentaose III, which is found at least on the parasite Schistosoma mansoni.

As used herein, the term “multivalent lacto-N-neotetraose” (multivalent LNnT) is intended to refer to a form of LNnT comprising multiple moieties of the carbohydrate, such as a form in which multiple LNnT carbohydrates are conjugated to a carrier molecule.

As used herein, the term “agent comprising LNnT” is intended to refer to a molecule or molecules that includes the LNnT carbohydrate moiety. In certain embodiments, the agent comprises LNnT with the proviso that the agent is not an antigen from S. mansoni, or an antigen from Toxocara canis, or is not Ascaris body fluid, or is not a filarial protein capable of inducing polyclonal IgE.

As used herein, the term “Lewis^{y} oligosaccharide” refers to a lacto type II carbohydrate comprising the structure: {Fuc(α1-2)Gal(β1-4)[Fuc(α1-3)]GlcNac}.

As used herein, the term “human immune cell” is intended to include cells of the human immune system which are capable of producing cytokines. Examples of human immune cells include human T cells, human macrophages and human B cells.

As used herein, the term “T cell” (i.e., T lymphocyte) is intended to include all cells within the T cell lineage, including thymocytes, immature T cells, mature T cells and the like, from a mammal (e.g., human or mouse).

As used herein, a “T helper type 2 response” (Th2 response) refers to a response by CD4⁺ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production).

As used herein, the term “a cytokine that regulates development of a Th2 response” is intended to include cytokines that have an effect on the initiation and/or progression of a Th2 response, in particular, cytokines that promote the development of a Th2 response. Preferred cytokines that are produced by the methods of the invention are IL-4, IL-5 and IL-10.

As used herein, the various forms of the term “modulation” are intended to include stimulation (e.g., increasing or upregulating a particular response or activity) and inhibition (e.g., decreasing or downregulating a particular response or activity).

As used herein, the term “contacting” (i.e., contacting an agent with a cell) is intended to include incubating the agent and the cell together in vitro (e.g., adding the agent to cells in culture) and administering the agent to a subject such that the agent and cells of the subject are contacted in vivo.

Various aspects of the invention are described in further detail in the following subsections.

I. Immunomodulatory Agents

In the immunomodulatory methods of the invention, a cell (e.g., a human immune cell, macrophage or T cell) is contacted either in vitro or in vivo with an agent such that an immune response is modulated. Preferably, the agent itself comprises LNnT, as described in further detail below. In one embodiment, the agent stimulates production by the cell of at least one cytokine (e.g., a cytokine that regulates development of a Th2 response). In another embodiment, the agent stimulates production of IL-4. In another embodiment, the agent stimulates cellular proliferation (e.g., B cell proliferation). In yet another embodiment, the agent stimulates production of non-specific polyclonal IgE.

A. Agents

The agents of the invention stimulate cytokine production by cells, stimulate production of non-specific polyclonal IgE by cells, and/or stimulate proliferation of cells. Accordingly, in one embodiment, the agent is a stimulatory form of a compound comprising LNnT. A “stimulatory form of a compound comprising LNnT” typically is one in which the carbohydrate structure (e.g., the LNnT) is present in a multivalent, crosslinked form. In a preferred embodiment, the stimulatory form of a compound comprising LNnT is a conjugate of a carrier molecule and multiple carbohydrate molecules (e.g., the LNnT). For example, carbohydrate molecules can be conjugated to a protein carrier, such as a conjugate of human serum albumin (HSA). When a sugar-carrier protein conjugate is to be administered to a subject, the carrier protein should be selected such that an immunological reaction to the carrier protein is not stimulated in the subject (e.g., a human carrier protein should be used with a human subject, etc). Alternative to a carrier protein, multivalent LNnT can be conjugated to other carrier molecules, for example carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Other preferred carriers include polymers, such as carbohydrate or polysaccharide polymers. A preferred carbohydrate polymer is dextran.

The degree of stimulatory ability of the conjugate is influenced by the density of sugars conjugated to the carrier. Preferably, the sugar molecules comprise at least 10% of the conjugate by weight, more preferably at least 15% of the conjugate by weight, even more preferably at least 20% of the conjugate by weight and even more preferably at least 25% of the conjugate by weight or at least 30% of the conjugate by weight or at least 35% of the conjugate by weight or at least 40% of the conjugate by weight or at least 45% of the conjugate by weight. In certain embodiments, the sugar molecules comprise about 10-25% of the conjugate by weight, about 15-25% of the conjugate by weight or about 20-25% of the conjugate by weight or about 30-35% by weight or about 35-40% by weight or about 40-45% by weight. In a preferred embodiment, the stimulatory form of a compound comprising LNnT is a conjugate of multiple carbohydrate molecules. More preferably, the conjugates comprise 10-11, 12-13, 14-15, 6-17, 18-19, or 20 or more sugars/conjugate. Agents for use in the methods of the invention can be purchased commercially or can be purified or synthesized by standard methods. Conjugates of LNnT and a carrier protein (e.g., HSA) are available from Accurate Chemical and Scientific Corporation, Westbury, N.Y.

In addition to conjugates comprising LNnT described above, another form of a stimulatory agent comprising LNnT is an isolated protein that naturally expresses LNnT in a form suitable for stimulatory activity.

The ability of an agent of the invention to stimulate production by immune cells of a cytokine can be evaluated using an in vitro culture system such as that described in the Examples. Cells are cultured in the presence of the agent to be evaluated in a medium suitable for culture of the chosen cells. After a period of time (e.g., 24, 48, 72, or 120 hours), production of the cytokine is assessed by determining the level of the cytokine in the culture supernatant. Preferably, the cytokine assayed is IL-4. Additionally or alternatively, IL-2, IL-5, IL-10, IL-13 and/or IFN-γ levels can be assessed. Cytokine levels in the culture supernatant can be measured by standard methods, such as by an enzyme linked immunosorbent assay (ELISA) utilizing a monoclonal antibody that specifically binds the cytokine. The ability of the agent to stimulate cytokine production is evidenced by a higher level of cytokine (e.g., IL-4) in the supernatants of cells cultured in the presence of the agent compared to the level of cytokine in the supernatant of cells cultured on the absence of the agent.

The ability of an agent of the invention to stimulate production of non-specific polyclonal IgE by cells (e.g., immune cells) can be evaluated in vitro utilizing methods such as those described in the Examples. For example, serum isolated from a subject can be analyzed by sandwich ELISA for the presence of total, as well as antigen-specific, IgE. Briefly, plates are coated with anti-IgE antibodies, washed extensively, blocked to prevent non-specific adsorption of reagents to the plate, then incubated with serum samples isolated from subjects. Labeled antibody (e.g., biotinylated anti-IgE antibody) can be used to detect antigen, for example, by detection with peroxidase-congugated strepavidin. The reactions can be subsequently developed using, for example, tetramethyl-benzidine substrate. Such methods are further useful for detection of, for example, Ag-specific IgG, HSA-specific IgE, LNnT-HSA-specific IgE, as well as specific IgG subtypes, by altering the specificity of the primary antibody (e.g., that used in initial coating of the plate).

The ability of an agent of the invention to stimulate proliferation of cells (e.g., proliferation responses) can be evaluated in vitro utilizing methods such as those described in the Examples. For example, spleen cells can be isolated from sacrificed mice, cultured in vitro in appropriate culture medium, and labeled with ³H thymidine as an indicator of DNA replication.

II. Pharmaceutical Compositions

Another aspect of the invention pertains to pharmaceutical compositions of the agents (e.g., stimulatory agents) of the invention. The pharmaceutical compositions of the invention typically comprise an agent of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, transdermal or oral administration. In a preferred embodiment, the composition is formulated such that it is suitable for intraperitoneal administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the modulators can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the agent may be coated in a material to protect it from the action of enzymes, acids and other natural conditions which may inactivate the agent. For example, the agent can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., a stimulatory agent of the invention) preferably is formulated in the composition in a therapeutically effective amount. A “therapeutically effective amount” refers to an amount effective, at dosages and for periods of time necessary, to achieve the desired therapeutic result, such as the production of sufficient levels of non-specific polyclonal IgE to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result, for example, influencing the production of sufficient levels of non-specific polyclonal IgE for prophylactic purposes. Typically, since a prophylactic dose is used in subjects prior to or at an earlier stage of disease, the prophylactically effective amount will be less than the therapeutically effective amount.

A non-limiting range for a therapeutically or prophylactically effective amounts of a stimulatory or inhibitory agent of the invention is 0.01 nM-20 mM. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An agent of the invention can be formulated into a pharmaceutical composition wherein the agent is the only active compound therein. Alternatively, the pharmaceutical composition can contain additional active compounds. For example, two or more agents may be used in combination. Moreover, an agent of the invention can be combined with one or more other agents that have immunomodulatory properties. For example, a stimulatory agent may be combined with one or more cytokines or adjuvants.

A pharmaceutical composition of the invention, comprising a stimulatory or inhibitory agent of the invention, can be administered to a subject to modulate immune responses (e.g., production of non-specific polyclonal IgE) in the subject. As used herein, the term “subject” is intended to include living organisms in which an immune response can be elicited, e.g., mammals. Examples of subjects include humans, dogs, cats, mice, rats, and transgenic species thereof.

A pharmaceutical composition of the invention can be formulated to be suitable for a particular route of administration. For example, in various embodiments, a pharmaceutical composition of the invention can be suitable for injection, inhalation or insufflation (either through the mouth or the nose), or for intranasal, mucosal, oral, buccal, parenteral, rectal, intramuscular, intravenous, intraperitoneal, and subcutaneous delivery.

In certain embodiments, a pharmaceutical composition of the invention can be packaged with instructions for using the pharmaceutical composition for a particular purpose, such as to modulate an immune response, for use as an adjuvant, to modulate an allergic response or to modulate an autoimmune disease.

III. Modulation of Immune Responses

The invention provides immunomodulatory methods that can be used modulate various immune responses. In the methods of the invention, a cell is contacted with an agent (e.g., an agent comprising LNnT) with the cell such that the immune response is modulated (i.e., stimulated). The methods of the invention can be practiced either in vitro or in vivo. For practicing the method of the invention in vitro, cells can be obtained from a subject by standard methods and incubated (i.e., cultured) in vitro with an agent of the invention to modulate, for example, the production of a cytokine, the production of non-specific, polyclonal IgE, proliferation of an immune cell (e.g., a splenocyte), or the development of a Th2 response. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, monocytes/macrophages can be isolated by adherence on plastic. T cells or B cells can be enriched or depleted, for example, by positive and/or negative selection using antibodies to T cell or B cell surface markers, for example by incubating cells with a specific mouse monoclonal antibody (mAb), followed by isolation of cells that bind the mAb using anti-mouse-Ig coated magnetic beads. Monoclonal antibodies to cell surface markers are commercially available.

For practicing the methods of the invention in vivo, an agent is administered to a subject in a pharmacologically acceptable carrier (as described in the previous section) in amounts sufficient to achieve the desired effect, such as to modulate, for example, the production of a cytokine, the production of non-specific, polyclonal IgE, proliferation of an immune cell, or the development of a Th2 response in the subject or to prevent a detrimental host reaction against parasite infection or to protect against environmental allergens by saturating FcεRs on effector cells in the subject or to inhibit a disease or disorder (e.g., an allergy or an autoimmune disease) in the subject. Any route of administration suitable for achieving the desired immunomodulatory effect is contemplated by the invention. One preferred route of administration for the agent is intraperitoneal. Another preferred route of administration is orally. Yet another preferred route of administration is intravenous. Application of the methods of the invention to the treatment of disease conditions may result in cure of the condition, a decrease in the type or number of symptoms associated with the condition, either in the long term or short term (i.e., amelioration of the condition) or simply a transient beneficial effect to the subject.

Numerous disease conditions associated with a predominant Th2-type response have been identified and could benefit from modulation of the type of response mounted in the individual suffering from the disease condition. Application of the immunomodulatory methods of the invention to such diseases as cancer, infectious disease, allergies, autoimmune disease, and inflammatory bowel disease. In addition to the foregoing disease situations, the immunomodulatory methods of the invention also are useful for other purposes. For example, the methods of the invention (i.e., methods using a stimulatory agent) can be used to stimulate production cytokines (such as IL-4) in vitro for commercial production of these cytokines (e.g., cells can be cultured with a stimulatory agent in vitro to stimulate IL-4 production and the IL-4 can be recovered from the culture supernatant, further purified if necessary, and packaged for commercial use).

This invention is further illustrated by the following examples which should not be construed as limiting. The contents of all references, patents and published patent applications cited throughout this application are hereby incorporated by reference.

### EXAMPLES

Materials and Methods Used in the Examples

Animals

Young adult (7-9 weeks old) CBA/J, BALB/C, and C57BL/6 strain female mice were purchased from Harlan (Indianapolis, Ind.). Female CBA/CaJ xid and age-matched control female CBA/CaJ mice were purchased from The Jackson Laboratory (Bar Harbor, ME). IL-4 deficient BALB/C mice were generated as described ( ). This deficient mice were bred and maintained at Harvard School of Public Health according to the guidelines set forth by the Harvard Medical Area Research Committee.

Antigens and Inoculations

Human serum albumin (HSA) was selected as a carrier protein for multivalent carbohydrate because HSA is a simple protein and does not contain any carbohydrate motif. Multivalent LNnT or Lewis^{γ} were conjugated with HSA (LNnT-HSA and Le^{y}-HSA) by Accurate Chemical and Scientific Corporation (NY). In both neoglycoproteins, 13 molecules of sugar conjugated to 1 HSA molecule. As controls, HSA (Sigma Chemical Co., Mo.), HSA adsorbed to alum (Intergen Company, NY; HSA-alum), or Dulbecco's PBS (Gibco BRL, NY) were prepared for immunization. Groups of four to six mice were immunized intraperitoneally or subcutaneously with Ags (10 μg of HSA) or saline. First and second boosting immunization were performed in the same fashion 2 and 3 weeks later, respectively. Protein concentration was determined by bicinchoninic acid (BCA) assay (Pierce, Ill.).

Determination of Serum Ab Titers

Immunized mice were bled from the tail 10, 6, and 5 days following primary, first, and second boosting immunization, respectively. Total and HSA-specific IgE were determined by sandwich ELISA. In brief, ELISA plates (Corning Inc., NY) were coated overnight at 4° C. with 100 μl of 5 μg/ml rat anti-mouse IgE mAb (Biosource, CA) in carbonate-bicarbonate buffer, pH 9.6. After washing four times with PBS containing 0.05% Tween20 (PBS-T), plates were blocked with 200 μl PBS containing 10% FCS and 0.3% Tween20 for 2 hrs at 37° C. After washing as above, 100 μl samples of serially-diluted serum or standard mouse IgEmAb (Pharmingen, Calif.) were added in duplicate wells and incubated for 2 hrs at 37° C. Thereafter, 100 μl biotinylated anti-mouse IgEmAb (0.5 μg/ml, Pharmingen) or biotinylated HSA (1 μg/ml) were added to detect total IgE and HSA-specific IgE, respectively. After 2 hrs incubation at 37° C., plates were washed and 100 μl peroxidase-conjugated streptavidin (Sigma) diluted 1/1000 was added to each well and incubated for 1 hr at 37° C. Finally plates were washed eight times, the reactions were developed by addition of tetramethyl-benzidine substrate (Kirkegaard and Perry Laboratories, Gaithersburg, Md.) and stopped by addition of phosphoric acid (0.4M). The absorbance was measured at 450 nm in UVMax automated plate reader (Molecular Devices Corp., Menlo Park, Calif.). For biotinylation, HSA (2 mg/ml) in sodium bicarbonate buffer pH 8.5 was incubated with biotin (long arm) N-hydroxy succumide ester (Vector Lab., CA) for 2 hrs at room temperature, stopped the reaction by addition of 5 μl ethanolamine, and dialyzed overnight with PBS/0.05% sodium azide.

Ag-specific IgG ELISA were determined. Briefly, ELISA plates were coated with 100 μl Ag (2 μg/ml) overnight at 4° C. in carbonate-bicarbonate buffer, and blocked as described above. Then plates were incubated with samples from individual serum in two-fold serial dilution from 100 times for 2 hrs at 37° C., followed by goat anti-mouse IgG mAb-peroxidase conjugate (Boehringer-Mannheim, Ind.) for 1 hr at 37° C. Thereafter, plates were developed and terminated as described above. Finally the absorbance at 450 nm was measured using a UVMax automatic microplate reader. Results were expressed as endpoint titers where the endpoint was determined as the final serum dilution which yields a higher absorbance than twice of the background absorbance. Optimum dilutions of anti-mouse IgG mAb-horseradish peroxidase-labeled conjugates were determined to be 1/1000. Plasma IgE specific for LNnT-HSA was also tested for in the same fashion as this method using LNnT-HSA (2 μg/ml) as a coating Ag and biotinylated anti-mouse IgEmAb (1 μg/ml, Pharmingen) followed by avidin-peroxidase conjugate (Sigma) as a detection Ab.

Serum total IgG isotypes were also determined by ELISA. Plates were coated with 100 μl of 2 μg/ml rat anti-mouse IgG1, IgG2a, IgG2b, and IgG3 mAb (Pharmingen) overnight at 4° C. After washing and blocking described as above, 100 μl samples of serially-diluted serum or standard mouse IgG isotypes (Pharmigen) were added in duplicate wells and incubated for 2 hrs at 37° C. Thereafter, 100 μl peroxidase-conjugated polyclonal goat anti-mouse Ig (Pharmingen) diluted in 1/1000 were added and incubated for 1 hr at 37° C. After the washing, the reactions were developed and stopped as described above, and the absorbance was measured at 450 nm.

Proliferative Responses of Splenocytes

Groups of mice were killed by carbon dioxide 5 days following second boosting immunization. Spleens were removed aseptically and proliferation assays were performed as described previously (Velupillai, P. et al. (1997) J. Immunol. vol. 158 pp. 338-344). In brief, cell suspensions were prepared in RPMI 1640 supplemented with 10% FCS (Gibco), 2 mM L-glutamine, 5×10⁻⁵ M 2-mercaptoethanol, 100 Unit/ml penicillin, and 100 μg/ml streptomycin (Sigma). Red blood cells were removed by incubation in Boyle's solution, and 2.5×10⁶ cells per ml were added to 96-well flat-bottom tissue culture plates (Corning) in triplicate for 72 hrs at 37° C., 5% C02 in air, with 2 μg/ml ConA (Vector), 10 μg/ml HSA or LNnT-HSA. For the final 8 hrs, cells were incubated with 1 μCi ³H thymidine (Amersham Life Science Inc., IL) and then harvested onto filter paper for scintillation counting.

Cytokine Assays

In flat-bottom 24-well culture plates (Corning), suspensions of 2.5×10⁶ cells per ml were cultured with ConA (2 μg/ml) or without restimulation at 37° C. in supplemented RPMI 1640 medium as described above. 24, 72 and 120 hrs later, the cell cultures were then centrifuged, and the culture supernatants were harvested and kept frozen (−80° C.) until assayed. Pelleted cells were resuspended then stained with mAbs coupled to FITC or PE for immunofluorescent staining. IL-2, IL-4, IL-5, IL-10 and IFN-γ levels in supernatants from ConA-stimulated or unstimulated splenocytes were measured by capture ELISA. In brief, Maxisorp microtitre plates (Nunc Laboratories Ltd., Denmark) were coated with 50 μl of capture Ab at 2.0 μg/ml (rat anti-mouse IL-5, IL-10 and IFN-γ from Pharmingen and rat anti-mouse IL-4 from Endogen, Mass.) in Carbonate-bicarbonate buffer by overnight incubation at 4° C. Wells were then washed with PBS-T four times and blocked by addition of 10% FCS in PBS (2 hours, 37° C.). 50 μl of culture supernatants and appropriate recombinant standards were then added to individual well in duplicate. For standard curves, recombinant IL-5 (0 to 5,000 pg/ml), IL-10 (0 to 25,000 pg/ml), IFN-γ (0 to 20,000 pg/ml) (Pharmingen, Calif.) and IL-4 (0 to 1,500 pg/ml) (Endogen, Mass.) were used in duplicate. Following overnight incubation at 4° C., the wells were washed and appropriate 50 μl/well biotinylated detection Ab at 1 μg/ml (rat anti-mouse IL-5, IL-10 or IFN-γ from Pharmingin and rat anti-mouse IL-4 from Endogen) were added. For the detection of bound biotinylated Ab, 50 μl of streptavidin-alkaline phosphatase conjugate (1/2000, Pharmingen) was added to each well for 45 minutes, then washed. Level of cytokine in the wells was visualized by addition of p-nitrophenyl phosphate (Sigma) in glycine buffer. Absorbance was measured at 405 nm using a UVMax automatic microplate reader.

Immunofluorescent Staining

Following in vitro culture without restimulation, splenocytes were resuspended and incubated on ice for 15 min with mAbs as follows: anti-CD45R/B220 (RA3-6B2), anti-B7-1 (16-10A 1), and anti-B7-2 (GL-1) or isotype-matched controls. All mAbs were either FITC- or PE-conjugated (Pharmingen). After washing with Hanks' balanced salt solution (Gibco) containing 0.05% sodium azide, flow cytometry analysis was performed by using a FACSCalibur flow cytometer and Cell Quest software (Becton Dickinson, CA). Dead cells were excluded from analysis on the basis of propidium iodide (Molecular Probes, OR) staining. Lymphocytes were gated according to the physical characteristics of forward and side scatter and at least 10,000 events were acquired.

Statistics

Statistical analysis was performed using the Student's unpaired t test. A value of p<0.05 was considered significant.

### Example 1

Induction of Polyclonal IgE by Multivalent LNnT in vivo

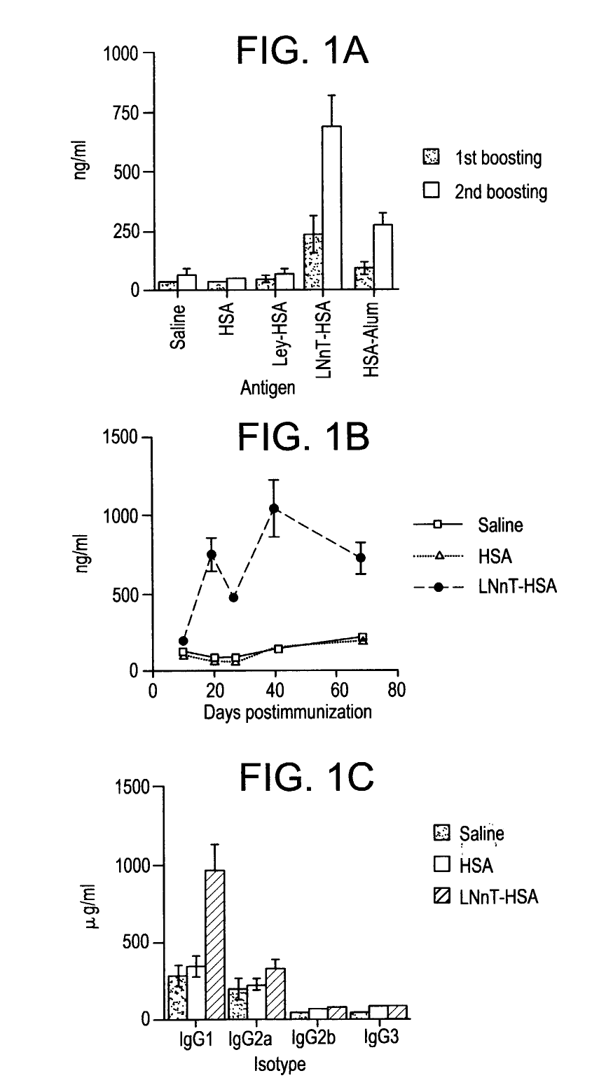

Following first boosting IP immunization with LNnT-HSA, BALB/C mice produced significantly higher amounts of serum total IgE than those IP immunized with saline, HSA, Le^{y}-HSA, and HSA-Alum (FIG. 1a). Serum IgE was significantly elevated following second IP immunization. This elevation was not seen following primary inoculation, however, high elevation of serum IgE lasted at least 8 weeks following second IP immunization with LNnT (FIG. 1b). Serum IgG1 was also significantly increased in mice IP immunized with multivalent LNnT compared with those immunized with saline or HSA alone, whereas the amount of other isotypes were not significantly different (FIG. 1c).

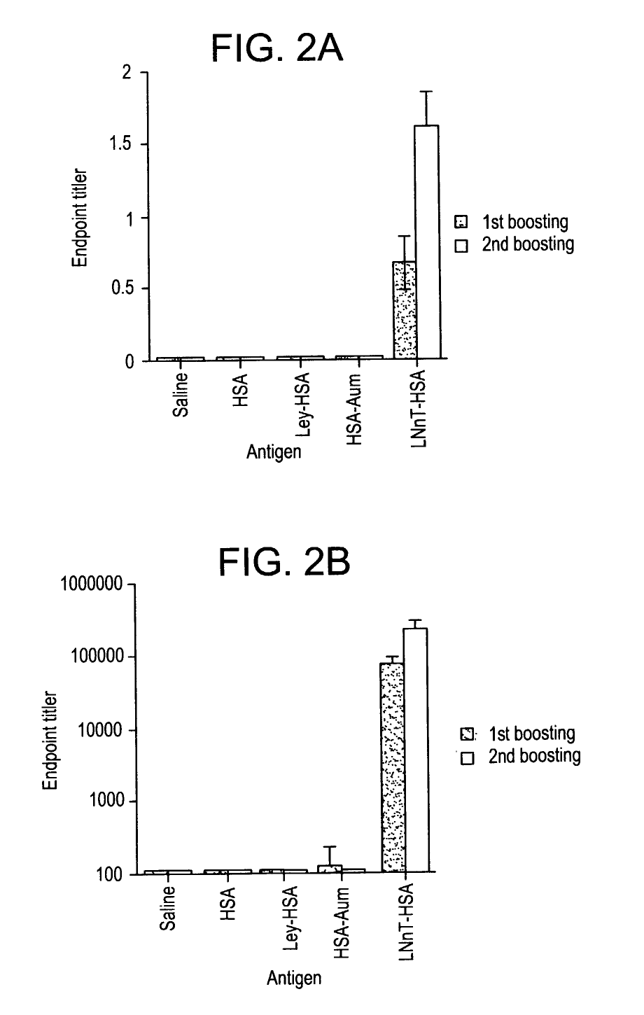

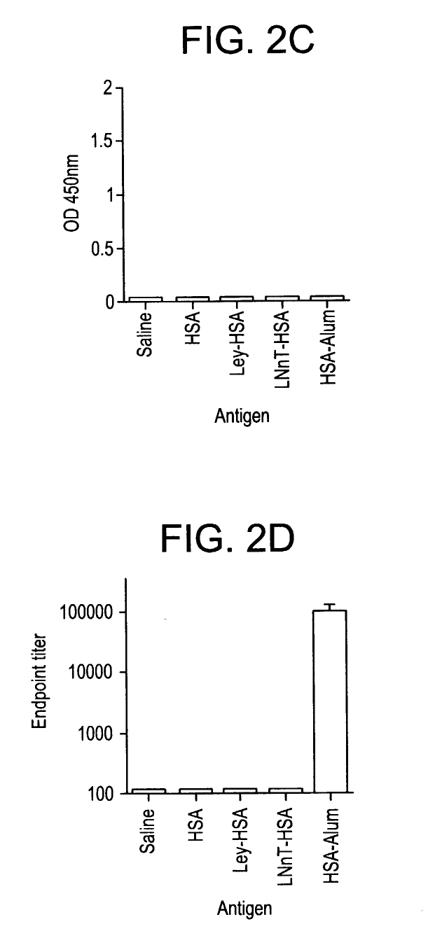

HSA-specific IgE and IgG were determined in sera in mice immunized IP with HSA-Alum. On the contrary, those signals in mice immunized IP with LNnT-HSA were not detected and were statistically no different from the control groups immunized with HSA alone, Le^{y}-HSA, or saline (FIGS. 2a,b). Similar findings were seen in Ab titers against LNnT-HSA. Mice IP immunized with LNnT-HSA did not produce significant amounts of IgG or IgE specific for LNnT-HSA compared with the controls (FIGS. 2c,d). Moreover, specific signal against Gal (β1-4) GlcNAc and Gal (β1-4) Glc, the components of LNnT, were also not detected when biotinylated Gal (β1-4) GlcNAc and Gal (β1-4) Glc (Glycotech, MD) were used, respectively, instead of biotinylated HSA in specific IgE ELISA. These results suggested that multivalent LNnT induced nonspecific polyclonal production of IgE in BALB/C mice following boosting immunization.

Specificity of Induction of Polyclonal IgE by Multivalent LNnT

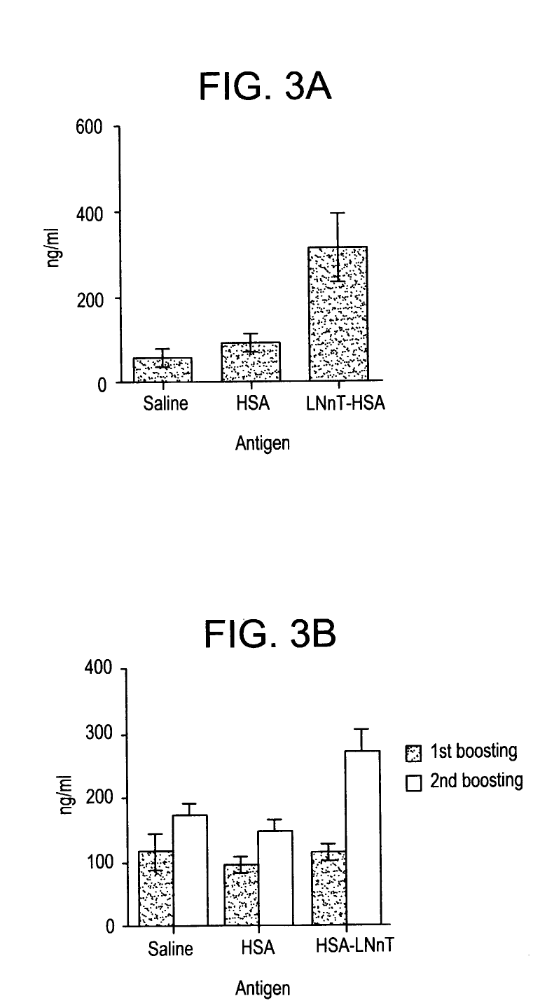

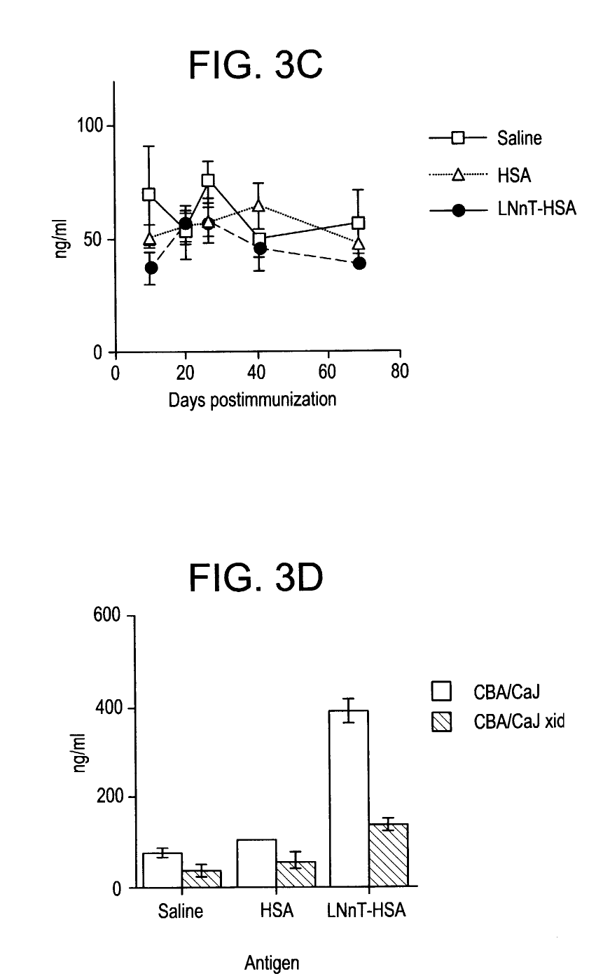

As in the BALB/C strain, CBA/J mice showed significant elevation of serum IgE following first boosting IP immunization with LNnT-HSA when compared to the control groups (FIG. 3a). On the other hand, C57BL/6 mice did not show the increase of serum IgE following first IP immunization with LNnT-HSA. However, this strain induced the increase of serum IgE following second IP immunization (FIG. 3b). Both strains did not elicit either IgE or IgG against HSA. These results indicate that the induction of polyclonal IgE by multivalent LNnT was genetically affected.

Elevation of serum total IgE was not seen in SC immunization with LNnT-HSA even following the boost (FIG. 3c). It has previously been suggested that LNnT-HSA does not induce such an elevation of serum IgE following intranasal sensitization, implying that the route of inoculation of multivalent LNnT is critical for the induction of nonspecific IgE. CBA/CaJ xid mice, which are deficient CD5+ B-1 cells, showed significantly higher amounts of IgE following IP immunization with LNnT-HSA compared with those IP immunized with saline or HSA, although the amounts were significantly lower than the control CBA/CaJ mice (FIG. 3d). This result indicates that B-1 cells, which is one of the specific components of peritoneal cavity, seems to be in part involved but not critical for the induction of polyclonal IgE by multivalent LNnT.

Proliferative Responses Against Multivalent LNnT

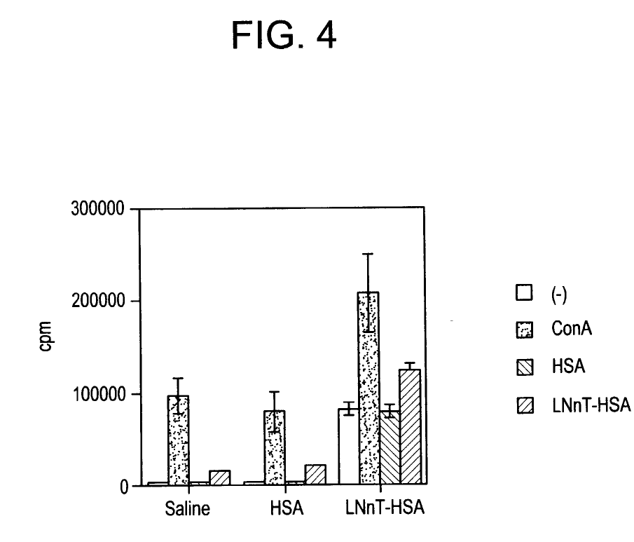

Splenocytes of BALB/C mice were prepared 5 days following second boosting IP immunization with saline, HSA or LNnT-HSA. Mice IP immunized with HSA or saline showed moderate but significant in vitro proliferative responses to LNnT-HSA compared to HSA or unrestimulation, suggesting that LNnT induces the proliferative responses in naive splenocytes. Mice immunized IP with LNnT-HSA showed the significant responses even without restimulation compared to the control mice (FIG. 4). In addition, the response was significantly enhanced with restimulation of LNnT-HSA In ConA stimulation, LNnT-inoculated mice also responded significantly more than the controls. These results suggested that splenocytes specific for LNnT-HSA were spontaneously activated following IP immunization with multivalent LNnT.

Cytokine Production by LNnT-Inoculated Mice

Splenocytes of mice IP immunized with LNnT-HSA also produced cytokines without restimulation. Significant amounts of IL-2, IL-4, and IL-5 were detected as early as 24 hours incubation in culture supernatants without restimulation. IL-10 and IFN-γ were detected as early as 72 hrs incubation. The results are summarized below in Table 1. Interestingly, in response to ConA, splenocytes of mice immunized IP with LNnT-HSA produced significantly more IL4, IL-5, and IL-10, but not IFN-γ compared to those IP immunized with saline or HSA, suggesting that LNnT skewed splenocytes into polarized Th2 responses in response to ConA stimulation.

TABLE 1

| | | in vitro stumulation (hrs) | | | |
|---|---|---|---|---|---|
| Cytokine | | None | None | None | ConA |
| (IP) | Immunization | (24 hrs) | (72 hrs) | (120 hrs) | (72 hrs) |
| IL-2 (pg/ml) | Saline | ND | ND | ND | ND |
| | HSA | ND | ND | ND | ND |
| | LNnT-HSA | 209 ± 68* | 2,060 ± 286* | 63 ± 1* | ND |
| IL-4 (pg/ml) | Saline | ND | ND | ND | 76.56 ± 18.28 |
| | HSA | ND | ND | ND | 31.81 ± 3.64 |
| | LNnT-HSA | 63.74 ± 24.11* | 479.07 ± 86.80* | 1,363.33 ± 259.70* | 234.00 ± 28.15* |
| IL-5 (pg/ml) | Saline | 48 ± 4 | 48 ± 2 | 49 ± 1 | 81 ± 2 |
| | HSA | 43 ± 2 | 42 ± 1 | 45 ± 6 | 78 ± 6 |
| | LNnT-HSA | 178 ± 5* | 808 ± 93* | 3,018 ± 604* | 671 ± 34* |
| IL-10 (pg/ml) | Saline | ND | ND | ND | ND |
| | HSA | ND | ND | ND | ND |
| | LNnT-HSA | ND | 1,068 ± 223* | 7,064 ± 1,381* | 1,529 ± 361* |
| IFN-g (pg/ml) | Saline | | | | |
| | HSA | ND | ND | ND | 3,577 ± 510 |
| | LNnT-HSA | ND | ND | ND | 3,358 ± 471 |
| | ND | 2,090 ± 891* | 12,220 ± 1,251* | 5,917 ± 1,132 | |

Selective in vitro B7-2 (CD86) Expression on B220+ Cells in LNnT-Inoculated Mice

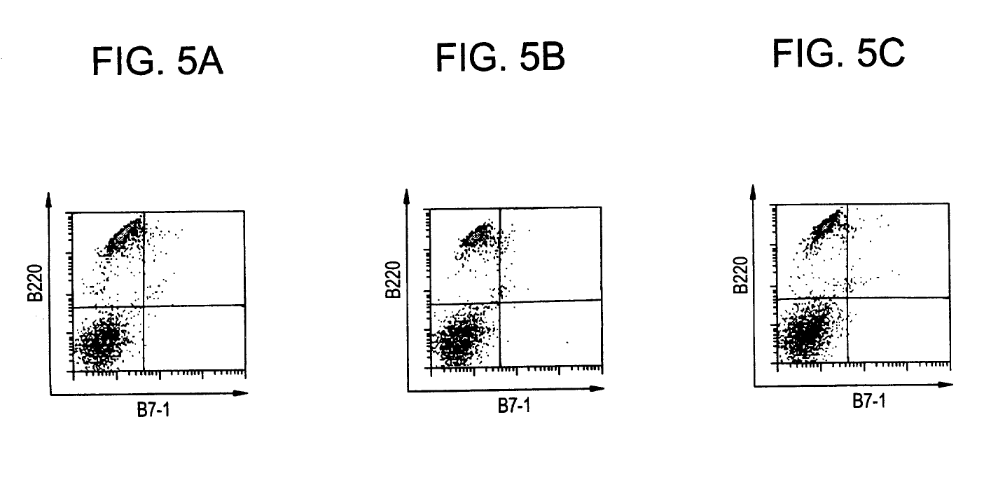

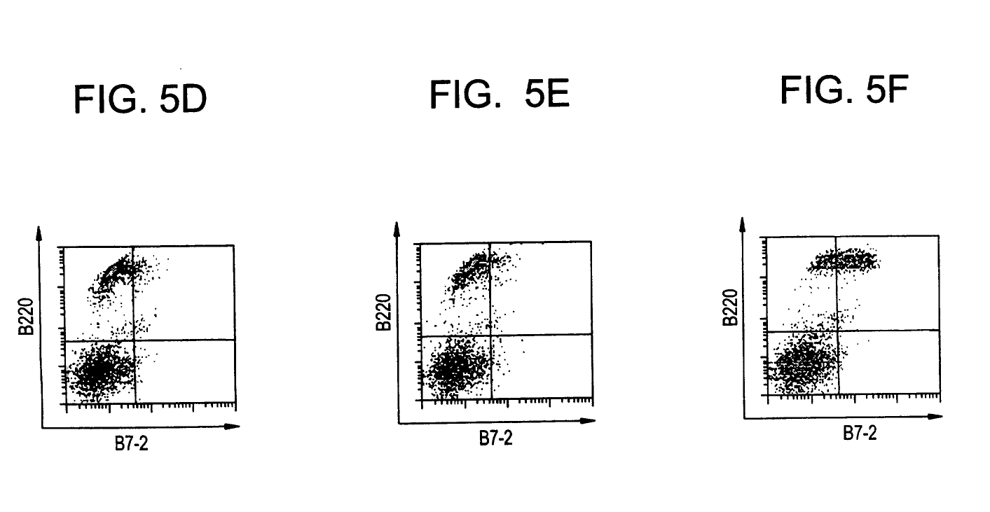

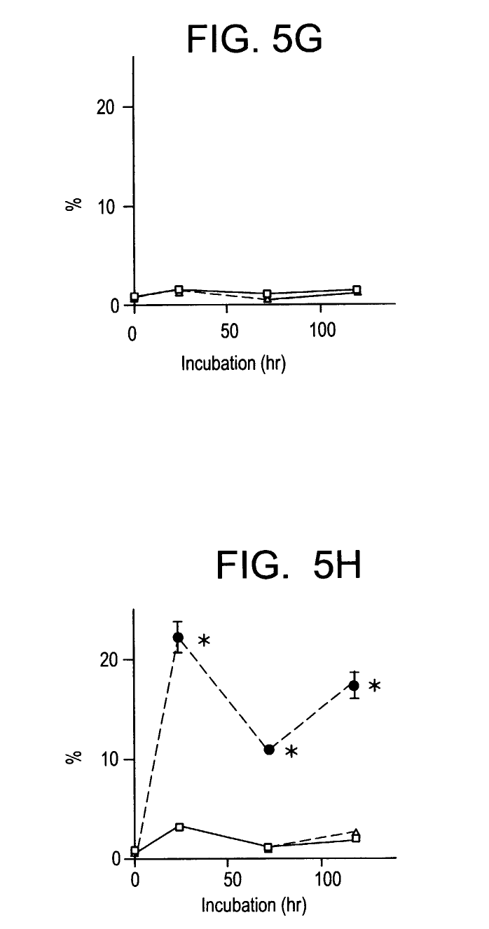

The expression of the costimulatory molecules, B7-1 and B7-2 on B220 positive splenocytes was also investigated. In freshly isolated splenocytes, the percentage of the cells positive for both B7-1 and B220 molecules were 1.02±0.08, 0.95±0.07, and 0.88±0.06 in mice IP immunized with saline, HSA, and LNnT-HSA, respectively (n=6). The percentage of the cells positive for both B7-2 and B220 molecules were 0.59±0.04, 0.59±0.05, and 0.56±0.04 in mice IP immunized with saline, HSA, and LNnT-HSA, respectively, suggesting no difference of the expression of these molecules in freshly isolated splenocytes. B7-1 expression was not altered in cultured splenocytes (FIGS. 5a,c,e). However, following 24 hrs culture incubation without restimulation in vitro, B220 positive cells expressing B7-2 molecule were increased in mice IP immunized with LNnT-HSA compared to the control mice immunized with saline or HSA (FIGS. 5b.d.f). This increase was found following 6 hr incubation, and lasted at least 120 hrs, although B7-1 expression on B220 positive cells was not altered throughout the period observed (FIGS. 5g,h).

IL-4 is Required for the Induction of Polyclonal IgE Production by Multivalent LNnT

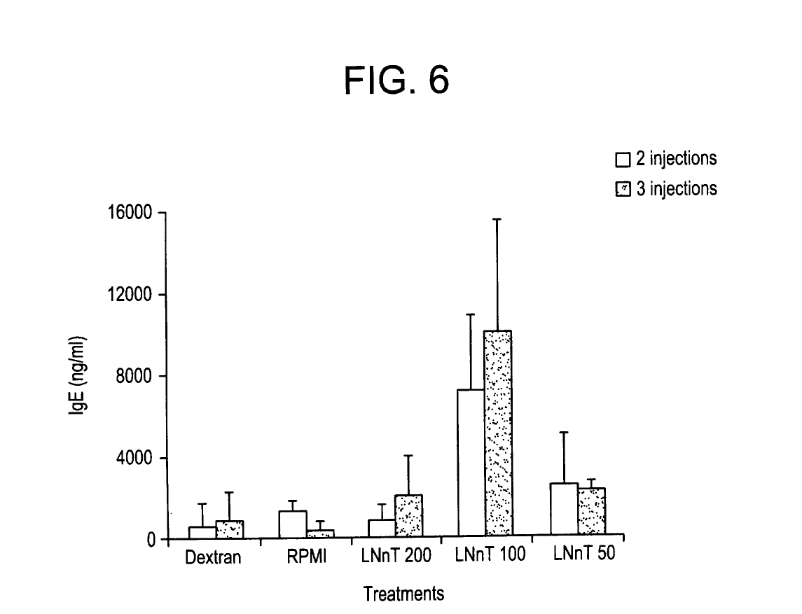

Because Th-2 cytokines, especially IL-4, are involved with IgE production, the role of IL-4 for the induction of polyclonal IgE production by multivalent LNnT in vivo was investigated using IL-4 gene deficient mice. IL-4 deficient mice did not induce polyclonal IgE production following repeated IP immunization with LNnT-HSA. In this experiment, total serum IgE. IL-4 deficient mice or wild type BALB/.C mice were immunized with saline, HSA or LNnT-HSA. Following second boosting immunization, sera were sampled and serum total IgE was measured. Following second boosting immunization, 2.5×10⁶/ml splenocytes from wild type or IL-4 deficient mice were cultured without additional stimulants and the production of IL-4, IL-5, IL-10, and IFN-γ were measured at 24, 72, and 120 hrs postincubation. Interestingly, splenocytes from IL-4 deficient mice with the immunization of LNnT-HSA produced IL-5, IL-6, and IFN-γ without restimulation. However, the amounts of IL-5 and IL-6 were significantly lower than wild type BALB/C mice (FIGS. 6b,c,d,e). These cytokines were not detected in IL-4 deficient mice IP immunized with saline or HSA, indicating that IL-4 is required for the induction of polyclonal IgE by multivalent LNnT.

### Example 2

Additional experiments demonstrating the induction of polyclonal IgE by multivalent LNnT were performed, as described below.

In a first series of experiments, mice were injected intraperitoneally either two or three times with either dextran alone, RPMI media or LNnT conjugated to dextran in saline (LNnT-dextran). The sugar conjugate was referred to as LNnT35, wherein the 35 refers to the degree of LNnT substitution on each dextran molecule. The total amount of IgE elicited in the mice was determined. The results are shown in FIG. 6, wherein the numbers after LNnT (i.e., 200, 100 or 50) refer to the amount injected (dextran weight). The results demonstrate that the LNnT-dextran construct is able to raise large amounts of total IgE in vivo.

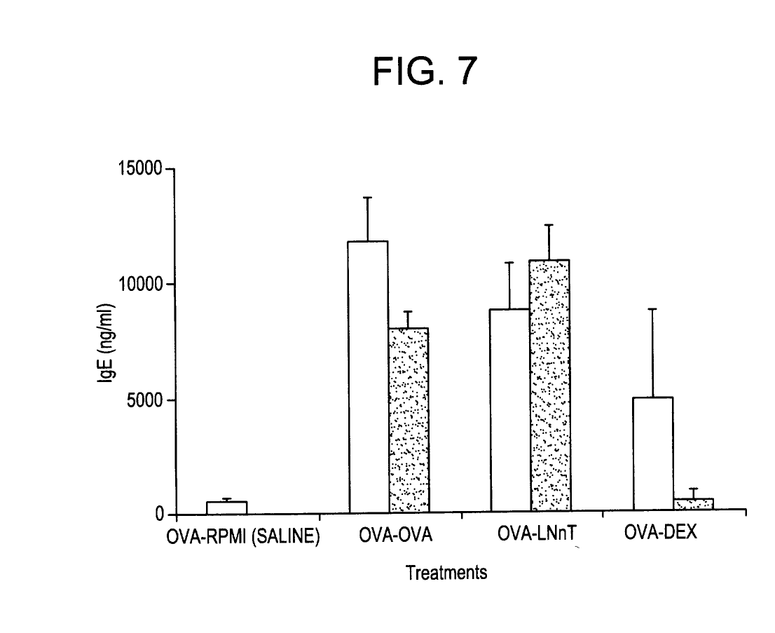

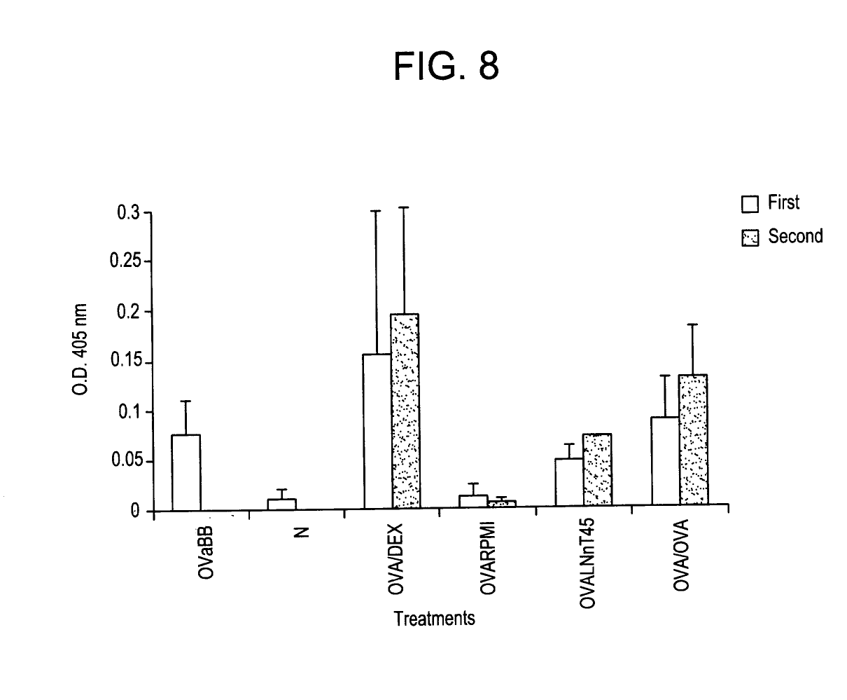

In another series of experiments, the effect of pre-treatment with multivalent LNnT on the production of antigen-specific IgE was investigated. Mice were injected either with ovalbumin (as a specific antigen) followed by a second injection of ovalbumin, or with the dextran conjugate LNnT45 followed by ovalbumin. Controls were mice receiving RPMI or dextran, followed by ovalbumin. FIG. 7 shows the total IgE in the mice and FIG. 8 shows the ovalbumin-specific IgE in the mice. FIG. 7 demonstrates that there was no discernible difference in levels of total IgE in the mice. FIG. 8 demonstrates, however, that prior treatment with the LNnT dextran conjugate does in fact reduce the amount of OVA-specific IgE by 40-50%. Thus, the LNnT conjugate functions to reduce the allergan specific IgE in vivo.

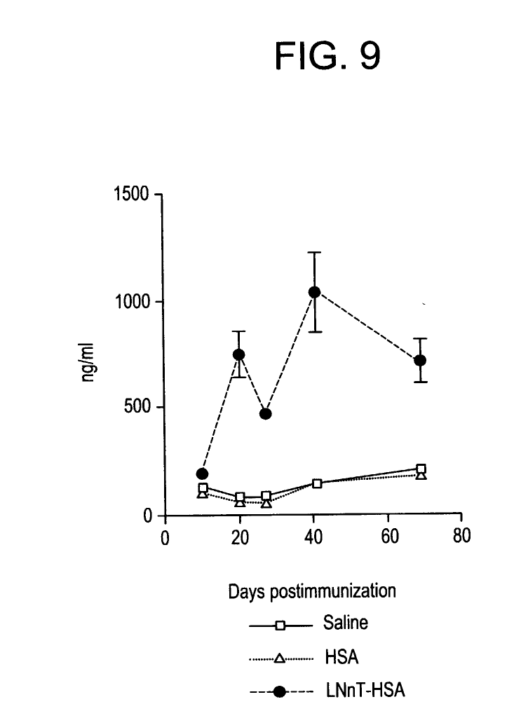

In another experiment, the length of time that non-specific IgE levels remain elevated after treatment with multivalent LNnT was investigated. LNnT-HSA was used as the conjugate, with saline and HSA alone serving as controls. The results are shown in FIG. 9, which demonstrates that mice treated with LNnT-HSA exhibited high levels of non-specific IgE for at least 70 days (the longest time point in the study). Thus, treatment with multivalent LNnT leads to persistent non-specific IgE.

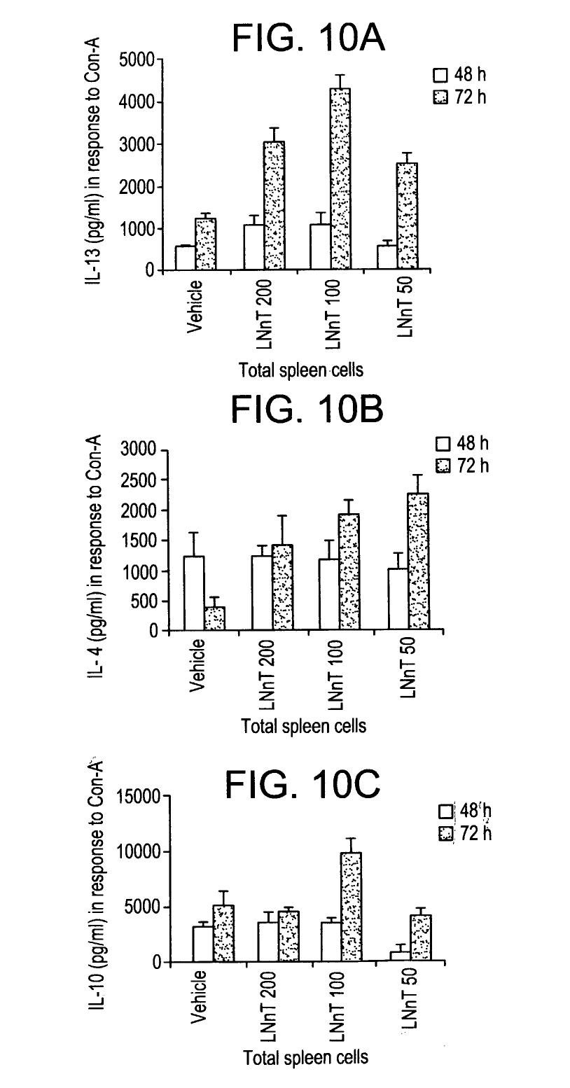

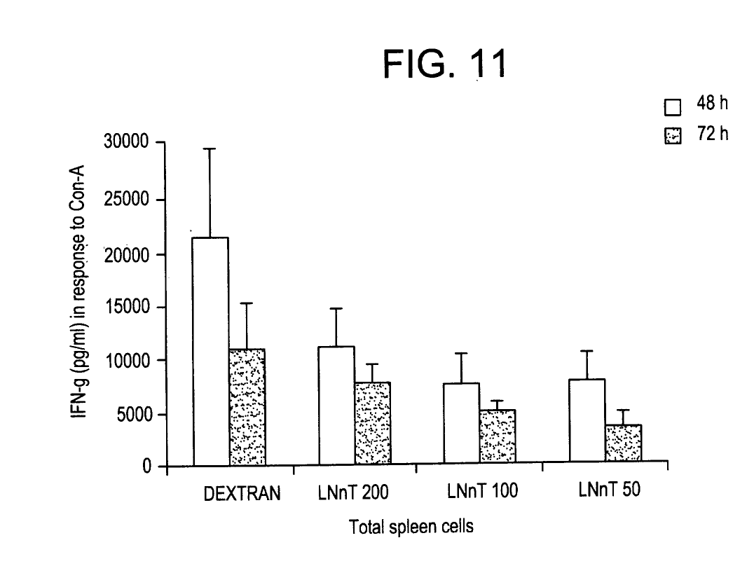

In another experiment, the effect of treatment with LNnT-dextran on production of either Th2-type cytokines or Th1-type cytokines was examined. Mice were immunized intraperitoneally with LNnT-dextran, total splenocytes were harvested and stimulated with ConA, followed by measurement of cytokine levels at 48 or 72 hours of culture. The results for Th2-type cytokines are shown in FIG. 10, which demonstrates that LNnT-dextran treatment leads to significantly elevated levels of IL-10, IL-4 and IL-13 at 72 hours culture compared to vehicle (dextran) immunized controls. The results for the Th1-type cytokine interferon-gamma are shown in FIG. 11, which demonstrates that LNnT-dextran treatment reduces the level of interferon-gamma at both 48 and 72 hours.

Discussion

These examples teach that multivalent LNnT induces polyclonal IgE production in mice by repeating IP immunization in vivo. Despite the fact that several reports have demonstrated polyclonal IgE production in mice and human in vivo and in vitro by the extracts from the parasites, the mechanism for promoting polyclonal IgE production by parasites is yet unclear (Wang, M Q. et al. (1995) Parasite Immunol. vol. 17 pp. 609-615; McGibbon, A M. et al. (1990) Mol. Biochem. Parasitol. vol. 39 pp. 163-172; Lee, T D G. et al. (1995) J. Allergy Clin. Immunol. vol. 95 pp. 124-1254Yarnashita, T. et al. (1993) Immunology vol. 79 pp. 185-195.Yamashita, U. et al. (1993) Jap. J. Parasitol. vol. 42 pp. 211-219). In fact, B cells are nonspecifically activated in parasite-infected host (Fisher, E. et al. (1981) Clin. Exp. Immunol. vol. 46 pp. ⁸⁹-97). And carbohydrate moieties on schistosoma japonicum egg antigen is reported to activate not only schistosome-primed but also naive B cells (Yamashita, T. et al. (1993) Immunology vol. 79 pp. 185-195). Therefore it seems that schistosomal antigens, especially carbohydrate moieties, have a mitogenic effect against B cells to induce nonspecific activation. Indeed, there are reports that the extracts from nematodes contain a B cell mitogen that regulate nonspecific B cell activation to induce polyclonal IgE production (Wang, M Q. et al. (1995) Parasite Immunol. vol. 17 pp. 609-615; Lee, T D G. et al. (1995) J. Allergy Clin. Immunol. vol. 95 pp. 124-1254). However, B cell mitogenic activity is not enough to promote polyclonal IgE production.

It is known that IL-4 induces B cells to develop polyclonal IgE producing cells in mice in vivo and in vitro (Coffman, R L. et al. (1986) J. Immunol. vol. 136 pp. 949-954; Finkelman, F D et al. (1990) Annu. Rev. Immunol. vol. 8 pp. 303-333; Tepper, R I. et al. (1990) Cell vol. 62 pp. 457; Snapper, C M. et al. (1991) J. Immunol. vol. 147 pp. 1163-1170; Nakanishi, K. et al. (1995) Int. Immunol. vol. 7 pp. 259-268). Therefore the help of IL-4 derived from IL-4 producing cells such as T cells (including NK1⁺ CD4⁺ T cells), eosinophils, and cells of the mast cell/basophil lineage may be required (Coffman, R L. et al. (1997) J. Exp. Med. vol. 185 pp. 373-375; Sabin, E A. et al. (1996) J. Exp. Med. vol. 184 pp. 1871-1878). Thus, at least two factors in parasite antigens may be required to induce polyclonal IgE production: B cell mitogenic activity and induction of IL-4 production. Lee et al. showed that the body fluid of Ascaris is capable of increasing total IgE levels in mice by a single subcutaneous injection (McGibbon, A M. et al. (1990) Mol. Biochem. Parasitol. vol. 39 pp. 163-172; Lee, T D G. et al. (1995) J. Allergy Clin. Immunol. vol. 95 pp. 124-1254; Lee, T D G. et al. (1993) Int. Arch. Allergy Immunol. vol. 102 pp. 185-190). However, they did not isolate the molecule that induce IgE production, but showed that ABA-1, the purified major allergen of Ascaris did not induce the increase of total IgE. Thus they suggested the model of polyclonal IgE induction by nematode that nematode products contain a B-cell mitogen that polyclonally activates B cells, which is converted into a polyclonal IgE response when these stimulated B cells come under the influence of IL-4 or an IL-4 like molecule activated by other factors.

Splenocytes from mice IP immunized with LNnT produced IL-4, IL-5, and IL-10 without restimulation in vitro, although they also produced detectable amount of IL-2 and IFN-γ. In addition, IL-4 deficient mice did not induce polyclonal IgE production following IP immunization with this carbohydrate although they produced significant amount of IL-5, IL-10, and IFN-γ. Development of IL-5 and IL-10 production following S. mansoni infection was also seen in IL-4 deficient mice (Pearce, E J. et al. (1996) Int. Immunol. vol. 8 pp. 435-444; King., C L. et al. (1996) Exp. Parasitol. vol. 84 pp. 245-252). These results indicate that IL-4 is required for inducing polyclonal IgE production by multivalent LNnT in vivo. Moreover, splenocytes from mice IP immunized with multivalent LNnT were found to produce significantly more IL-4 in response to ConA, the T cell mitogen. This result is consistent with the reports that schistosome-infected host shows mitogen-driven IL-4 production and the production is correlated to serum total IgE (King, CL. et al., (1993), J. Immunol. vol. 150 pp. 1873-1880; Ogilvie BM Nature 1964 204. 91-92; Zwingenberger, K. et al. (1991) Scand. J. Immunol. vol. 34 pp. 243-251). Therefore, multivalent LNnT likely skews host susceptible to produce IL-4. The source of IL-4 was analyzed by intracellular staining in in vitro culture, and it was demonstrated that CD4+ T cells are responsible for the production of IL-4.

Collecting these findings, multivalent LNnT may possess at least two functions, B cell mitogenic activity and induction of IL-4 production, to induce polyclonal IgE production. In fact, splenocyte from control mice IP immunized with saline also showed the significant proliferative responses against LNnT, suggesting that this carbohydrate possesses the mitogenic activity.

CBA/J and BALB/C mice produce the polyclonal IgE following second immunization, on the other hand, C57BL/6 mice produce it following third immunization. It is known that host response against S. mansoni infection is strain dependent. CBA/J, C3H/HeJ, and BALB/C mice developed bigger liver granulomas and higher portal hypertension whereas C57BL/6 mice developed relatively smaller granulomas and lower portal hypertension (Fanning, M M. et al. (1981) J. Inf. Dis. vol. 144 148-153; Hernandez, N J. et al. (1997) Eur. J. Immunol. vol. 27 pp. 666-670). Immunization with LNnT seems to have a same characteristics as S. mansoni infection in terms of the susceptible strain of mice, suggesting that this carbohydrate may be a dominant putative antigen in S. mansoni. Amiri et al. demonstrated that complete suppression of the total IgE response resulted in the decreases in worm burden and egg production in S. mansoni-infected normal and IFN-γ knockout mice (Amiri, P. et al. (1993) J. Exp. Med. vol. 180 pp. 43-51). The present result may relate with these reports that in primary S. mansoni infection, C57BL/6 mice produced relatively lower amount of polyclonal IgE production in response to carbohydrate antigen in S. mansoni, results in the decreased egg production and worm burden (Amiri, P. et al. (1992) Nature vol. 356 pp. 604).

In addition, it has been previously demonstrated that peritoneal B-1 cell outgrowth due to S. mansoni infection was strain dependent, occurring in CBA/J, C3H/HeJ, and BALB/C mice but not in C57BL/6 mice (Palanivel, V. et al. (1996) Exp. Parasitol. vol. 84 pp. 168-177). B-1 cell subset is a major source of B cell IL-10 that downregulate Th1 responses (Amiri, P. et al. (1992) Nature vol. 356 pp. ⁶⁰⁴). The present result that peritoneal B-1 cells seem to be involve in part in the induction of polyclonal IgE by multivalent LNnT (FIG. 2d) is consist with the report.

Polyclonal IgE production in response to multivalent LNnT is not due to LPS contamination, because mice IP immunized with LNnT-HSA produced same amount of serum IgG3 as compared with controls. It is known that LPS induced IgG3 production both in vivo and in vitro (Coffman, R L. et al. (1986) J. Immunol. vol. 136 pp. 949-954; Finkelman, F D et al. (1990) Annu. Rev. Immunol. vol. 8 pp. 303-333). And LPS itself did not induce IL-4 production in vitro, whereas splenocytes from mice IP immunized with LNnT-HSA did produce IL-4.

Following in vitro culture incubation without restimulation, B7-2 positive cells were increased in B220+ cell population in mice IP immunized with LNnT-HSA compared to the control mice immunized with saline or HSA. B7-1 and B7-2 costimulatory molecules are ligands for CD28/CTLA-4 and involved in T cell activation, cytokine production, and regulation of tolerance (McKnight, A J. et al. (1994) J. Immunol. vol. 152 pp. 5220-5225; Perez, V L. et al. (1997) Immunity vol. 6 pp. 411-417). Costimulation by B7-1 and B7-2 can differentially regulate Th1 cell differentiation, although the effect of these molecules are dependent on the status of immune reaction, doses and routes of antigen inoculation, types of APC, and the experimental model of diseases (Thompson, C B. (1995). Cell vol. 81 pp. 979-982). For example, in studies of experimental allergic encephalomyelitis (EAE) in mice, administration with anti-B7-1 diminished the severity of neurologic disease, which is mediated by Th1 cells, while anti-B7-2 administration enhanced the severity (Kuchroo, V K. et al. (1995) Cell vol. 80 pp. 707-718). Recent reports suggest that B7-2 may play a critical role in the ability to initiate a Th2 response (Thompson, C B. (1995). Cell vol. 81 pp. 979-982; McArthur, J G. et al. (1993) J. Exp. Med. vol. 178 pp. 1645-1653). The present results are consistent with these reports and suggest that B7-2 expression is closely associated with polyclonal IgE production by multivalent LNnT. However, freshly isolated B220+ cells from mice IP immunized with multivalent LNnT did not express the significant levels of B7-2 compared with those from control mice. This result means that LNnT indirectly induce the B7-2 expression on B220+ cells. This may be due to IL-4 secreted by Th2 cells, because IL-4 deficient mice did not induce B7-2 expression. Stack et al. demonstrated that IL-4 treatment of small splenic B cells induced both B7-2 and B7-2 molecules (Stack, R M. et al. (1994) J. Immunol. vol. 152 pp. 5723-5733. They also reported that B7-2 expression was detected at 6 hr and appeared to be maximal at 24 hr, whereas B7-1 was not observed until 48 hr and was maximal at 72 hr. B7-1 expression could not be detected until 120 hr. This difference may be due to the status of B cells. This result represents the expression of primed B220+ cells by multivalent LNnT, on the contrary, the authors investigated the resting B cells.

The current findings suggest that in vivo induction of polyclonal IgE production by multivalent LNnT may be useful for immunotherapy or prophylaxis of allergic and anaphylactic reaction in which detrimental chemical mediators are released from effector cells by the crosslink of antigen-specific IgE on the cell surface. Further, these results encourage us to apply for the reduction of anaphylactic reaction in not only parasite infection but also environmental allergy (Hagel, I. et al. (1993) Parasite Immunol. vol. 15 pp. 311-315).

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of modulating an immune response in a subject comprising: administering to the subject an agent comprising multivalent lacto-N-neotetraose (LNnT), such that an immune response is modulated in the subject.

2. The method of claim 1, wherein the agent comprises LNnT conjugated to a protein carrier.

3. The method of claim 2, wherein the agent comprises LNnT conjugated to human serum albumin.

4. The method of claim 1, wherein the agent comprises LNnT conjugated to a carbohydrate polymer.

5. The method of claim 4, wherein the agent comprises LNnT conjugated to dextran.

6. The method of claim 1, wherein the agent is administered intraperitoneally.

7. The method of claim 1, wherein the agent is administered intravenously.

8. The method of claim 1, wherein the immune response that is modulated is an IgE response.

9. The method of claim 8, wherein a polyclonal IgE response is stimulated.

10. The method of claim 8, wherein the method comprises administering the agent to the subject prior to exposure of the subject to an allergan and wherein production of an allergan-specific IgE response in the subject is inhibited.

11. A method of stimulating production of a Th2-type cytokine comprising contacting a cell capable of producing a Th2-type cytokine with an agent comprising multivalent lacto-N-neotetraose (LNnT) such that a Th2-type cytokine is produced by the cell.

12. The method of claim 1, wherein the cytokine is IL-4.

13. The method of claim 11 wherein the cytokine is IL-10 or IL-13.

14. The method of claim 11, wherein the agent comprises LNnT conjugated to a protein carrier.

15. The method of claim 14, wherein the agent comprises LNnT conjugated to human serum albumin.

16. The method of claim 11, wherein the agent comprises LNnT conjugated to a carbohydrate polymer.

17. The method of claim 16, wherein the agent comprises LNnT conjugated to dextran.

18. A method of stimulating proliferation of splenocytes comprising contacting splenocytes with an agent comprising multivalent lacto-N-neotetraose (LNnT) such that proliferation of the splenocytes is stimulated.

19. The method of claim 18, wherein the agent comprises LNnT conjugated to a protein carrier.

20. The method of claim 19, wherein the agent comprises LNnT conjugated to human serum albumin.

21. The method of claim 18, wherein the agent comprises LNnT conjugated to a carbohydrate polymer.

22. The method of claim 21, wherein the agent comprises LNnT conjugated to dextran.

23. A pharmaceutical composition comprising an agent comprising multivalent lacto-N-neotetraose (LNnT) and a pharmaceutical carrier, packaged with instructions for use of the pharmaceutical composition as a modulator of IgE responses in a subject.

24. The pharmaceutical composition of claim 23, wherein the agent comprises LNnT conjugated to a protein carrier.

25. The pharmaceutical composition of claim 24, wherein the agent comprises LNnT conjugated to human serum albumin.

26. The pharmaceutical composition of claim 23, wherein the agent comprises LNnT conjugated to a carbohydrate polymer.

27. The pharmaceutical composition of claim 26, wherein the agent comprises LNnT conjugated to dextran.
